(19) United States
(12) Patent Application Publication
(10) Pub. No.: US 2012/0226152 A1
(43) Pub. Date: Sep. 6, 2012

(54) Tumor Tracking System and Method for Radiotherapy

(76) Inventor: Fatih M. Porikli, Watertown, MA (US)

(21) Appl. No.: 13/039,906

(22) Filed: Mar. 3, 2011

(51) Int. Cl. A61B 6/00

(52) U.S. Cl. 600/427

(57) ABSTRACT

A system and method for tracking a tumor includes a regression module for selecting, using a motion signal and a regression function, a feature signal from a set of feature signals, each feature signal in the set of feature signals represents a medical image of the body of the patient, wherein the motion signal represents a motion of a surface of a skin of the patient caused by the respiration, and wherein the regression function is trained based on a set of observations of the motion signal synchronized with the set of feature signals; and a registration module for determining the location of the target object using the feature signal and a registration function, wherein the registration function registers each feature signal to a breath-hold location of the target object identified.

### FIELD OF THE INVENTION

This invention relates generally to radiotherapy, and more particularly to tracking a motion of a pathological anatomy during respiration of a patient while delivering particle beam radiotherapy.

### BACKGROUND OF THE INVENTION

One challenge during the delivery of radiation to a patient for treating a pathological anatomy, such as a tumor or a lesion, is identifying a location of the tumor. The most common localization methods use an x-ray to image the body of the patient to detect the location of the tumor. Those methods assume that the patient is stationary. However, even if the patient is stationary, radiotherapy requires additional methods to account for the motion of the tumor due to respiration, in particular for treating a tumor located near the lungs of the patient, e.g., posterior to the sternum. Breath-holding and respiratory gating are two primary methods used to compensate for the motion of the tumor during the respiration, while the patient receives the radiotherapy.

A breath-hold method requires that the patient holds the breath at the same time and duration during the breathing cycle, e.g., a hold of 20 seconds after completion of an inhalation, thus treating the tumor as stationary. A respirometer is often used to measure the rate of respiration, and to ensure the breath is being held at the same time in the breathing cycle. That method can require training the patient to hold the breath in a predictable manner.

Respiratory gating is the process of turning the beam on and off as a function of the breathing cycle. The radiotherapy is synchronized to the breathing pattern, limiting the radiation delivery to during a specific time of the breathing cycle and targeting the tumor only when the location of the tumor in a predetermined range. The respiratory gating method is usually quicker than the breath-hold method, but requires the patient to have many sessions of training to breathe in the same manner for long periods of time. Such training requires can require days of practice before treatment can begin. Also, with the respiratory gating some healthy tissue around the tumor can be irradiated to ensure complete treatment of the tumor.

Attempts have been made to avoid the burden placed on a patient treated by the breath-hold and respiratory gating methods. For example, one method tracks the motion of the tumor during the respiration using a combination of internal imaging markers and external position markers to detect the motion of the tumor. In particular, fiducial markers are placed near the tumor to monitor the tumor location. The position of the fiducial markers is coordinated with the external position markers to track the motion of the tumor. Because exposing the patient continuously to x-rays to monitor the position of fiducial markers is undesirable, the position of the external markers are used to predict the position of the fiducial markers between longer periods of x-raying. One type of the external position markers integrates light emitting diodes (LEDs) into a vest that is worn by the patient. The flashing LEDs are detected by a camera to track the motion. However, placement of the internal imaging markers near the organs of the patient is undesirable for number of medical and health related reasons.

### SUMMARY OF THE INVENTION

It is an object of the subject invention to provide a system and method for determining a location of a tumor in a body of a patient based on a motion of the skin of the patient.

It is further object of the invention to provide such a method that the location of the tumor is tracked during respiration of the patient.

It is further object of the invention to facilitate radiotherapy during any phase of the respiration of the patient.

It is further object of the invention to minimize tumor position uncertainty during the treatment.

It is further object of the invention to determine the location of the tumor without using invasive fiducial markers.

It is further object of the invention to minimize exposure of the patient to the unhealthy medical imaging, e.g., x-rays, during the treatment.

Embodiments of the invention are based on a realization that there is a correspondence between a motion of the skin surface due to the respiration of the patient and the motion of the tumor caused by the respiration. However, any implementation employing this realization faces numerous challenges.

Particularly, during an alignment of the patient during treatment sessions, reference x-ray images are taken for breath-in and hold times, referred herein as a “breath-hold,” to minimize the discrepancy with a tomography acquired during planning sessions. However, the x-ray and the tomography are acquired at different times, often weeks or months apart. Between the treatment and planning sessions, breath-hold pattern of the patient can change. The patient may gain or loose weight and organs might be shifted due to, e.g., fluid motion and/or gas.

Accordingly, the correspondence between the motion of the surface of the skin and the motion of the tumor has to be established during each treatment session. To that end, conventional methods use either invasive fiducial markers and/or an excessive four-dimensional imaging data of a body of the patient. Both of those methods are harmful to the patient. In some situations, the complex and time consuming data models are determined for each of the treatment session, which extend the time of the treatment and increase the potential damage to the health of the patient. However, that was not considered as a problem, but rather an inherent characteristic of the particle beam therapy.

Embodiments of the invention are based on another realization that the certain correspondences between the motion of the surface of the skin and the tumor can be determined during treatment planning sessions, and be reused multiple times during the treatment delivery sessions, thus reducing the time of treatment, and the unnecessary harm to the health of the patient.

Moreover, those correspondences can be updated during the treatment preparation sessions with images having lower quality than the images required to determine the correspondence. Thus, the treatment time and the risk of potential harm are reduced without compromising the quality of the correspondence.

Accordingly, one embodiment of the invention discloses a system for facilitating an operation of a treatment delivery system based on a location of a target object in a body of a patient, wherein the location is subject to a motion caused by respiration of the patient, comprising: a regression module for selecting, using a motion signal and a regression function, a feature signal from a set of feature signals, each feature signal in the set of feature signals represents a medical image of the body of the patient, wherein the motion signal represents a motion of a surface of a skin of the patient caused by the respiration, and wherein the regression function is trained based on a set of observations of the motion signal synchronized with the set of feature signals; a registration module for determining the location of the target object using the feature signal and a registration function, wherein the registration function registers each feature signal to a three-dimensional (3D) imaging data having a breath-hold location of the target object identified; and a control module for generating a command to facilitate the operation of the treatment delivery system based on the location.

Another embodiment discloses a method for controlling an operation of a treatment delivery system such that a beam of radiation is directed at a target object in a body of a patient for a duration of treatment session, wherein a location of the target object is subject to a motion caused by a respiration of the patient, comprising the steps of: selecting, using a motion signal and a regression function, a feature signal from a set of feature signals, each feature signal in the set of feature signals represents a medical image of the body of the patient, wherein the motion signal represents a motion of a surface of a skin of the patient caused by the respiration, and wherein the regression function is trained based on a set of observations of the motion signal synchronized with the set of feature signals; determining the location of the target object using the feature signal and a registration function, wherein the registration function registers each feature signal to a digitally reconstructed radiograph (DRR) image having a breath-hold location of the target object identified; and controlling the operation of the treatment delivery system based on the location, such that a beam of radiation is directed at the location of the target object.

Yet another embodiment discloses a system for facilitating an operation of a treatment delivery system based on a location of a tumor in a body of a patient, wherein the location is subject to a motion caused by respiration of the patient, comprising: a regression module for selecting, using a motion signal and a regression function, a feature signal from a set of feature signals, each feature signal in the set of feature signals represents a medical image of the body of the patient, wherein the motion signal represents a motion of a skin of the patient caused by the respiration, and wherein the regression function is trained based on a set of observations of the motion signal synchronized with the set of feature signals; a registration module for determining the location of the tumor using the feature signal and a registration function, wherein the registration function registers each feature signal to a breath-hold location of the tumor; and an update module for updating the regression function and the registration function based on a subset of feature signals representing different locations of the tumor.

### DEFINITIONS

In describing embodiments of the invention, the following definitions are applicable throughout.

A “computer” refers to any apparatus that is capable of accepting a structured input, processing the structured input according to prescribed rules, and producing results of the processing as output. Examples of a computer include a computer; a general-purpose computer; a supercomputer; a mainframe; a super mini-computer; a mini-computer; a workstation; a microcomputer; a server; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

A “central processing unit (CPU)” or a “processor” refers to a computer or a component of a computer that reads and executes software instructions.

A “memory” or a “computer-readable medium” refers to any storage for storing data accessible by a computer. Examples include a magnetic hard disk; a floppy disk; an optical disk, like a CD-ROM or a DVD; a magnetic tape; a memory chip; and a carrier wave used to carry computer-readable electronic data, such as those used in transmitting and receiving e-mail or in accessing a network, and a computer memory, e.g., random-access memory (RAM).

“Software” refers to prescribed rules to operate a computer. Examples of software include software; code segments; instructions; computer programs; and programmed logic. Software of intelligent systems may be capable of self-learning.

A “module” or a “unit” refers to a basic component in a computer that performs a task or part of a task. It can be implemented by either software or hardware.

A “control system” refers to a device or a set of devices to manage, command, direct or regulate the behavior of other devices or systems. The control system can be implemented by either software or hardware, and can include one or several modules.

### BRIEF DESCRIPTION OF THE DRAWINGS

FIG. 1 is a schematic of a treatment delivery system for performing radiotherapy according to one embodiment of the invention;

FIG. 2 is a block diagram of a system and a method for determining a location of a tumor in a body of a patient according to one embodiment of the invention;

FIG. 3 is a block diagram of a method for determining a global location of the tumor according to one embodiment of the invention;

FIGS. 4A-B are schematics of a determining signal of a motion of the skin of a patient;

FIG. 5 is a schematic of training a regression function;

FIG. 6 is a block diagram of a method for determining the regression function according to one embodiment of the invention;

FIG. 7 is a block diagram of a method for determining a registration function according to one embodiment of the invention;

FIG. 8 is a block diagram of a method for updating the regression and the registration function based on a subset of medical images according to one embodiment of the invention;

FIG. 9 is a block diagram of a method for determining a subset of medical images according to one embodiment of the invention;

FIG. 10 is a block diagram of updating the regression function according to one embodiment of the invention; and

FIG. 11 is a block diagram of updating the registration function according to one embodiment of the invention; and

FIG. 12 is a schematic of different procedures the embodiments of the invention are taken advantage form.

### DETAILED DESCRIPTION OF THE PREFERRED EMBODIMENT

The radiotherapy treatment procedure typically includes multiple sessions such as a treatment planning session, a treatment preparation session and a treatment delivery session. Each session includes one or more procedures depending on a particular medical condition of a patient.

Treatment Planning Session

During the treatment planning session, data of the patient are acquired and an appropriate beam radiotherapy treatment procedure is planned including, but not limited to determination of the patient treatment position, identification of the target volumes and organs at risk, determination and verification of the treatment field geometry, and generation of simulation radiographs for each treatment beam.

The entire process of treatment planning involves many steps and usually involves a radiation oncology team, including an oncology physician, a clinical physicist, and a dosimetrist. The team is responsible for the overall integrity of the system to accurately and reliably produce dose distributions and associated calculations for radiotherapy.

Typically, medical imaging, e.g., computed tomography, magnetic resonance imaging, and positron emission tomography, is used to form a virtual patient for a computer-aided design procedure. Treatment simulations are used to plan the geometric and radiological aspects of the therapy using radiation transport simulations and optimization. Plans are often assessed with the aid of dose-volume histograms, allowing a clinician to evaluate the uniformity of the dose to the diseased tissue, e.g., tumor and sparing of healthy structures.

Computerized treatment planning systems are used to generate beam shapes and dose distributions with the intent to maximize tumor control and minimize normal tissue complications. Patient anatomy and tumor targets can be represented as 3-D models.

Treatment Preparation Session

The treatment preparation session is typically performed immediately before every treatment session. During the treatment preparation session, the patient is prepared for the treatment, positioned on the treatment couch and a pose of the patient is aligned using automated image registration tools. Also, auxiliary data of the patient are recorded.

Treatment Delivery Session

During the treatment delivery session, the patient is given the prescribed dose of the radiation. As described in more details below, embodiments of the invention track the motion of the tumor to enable continuous radiation.

System Overview

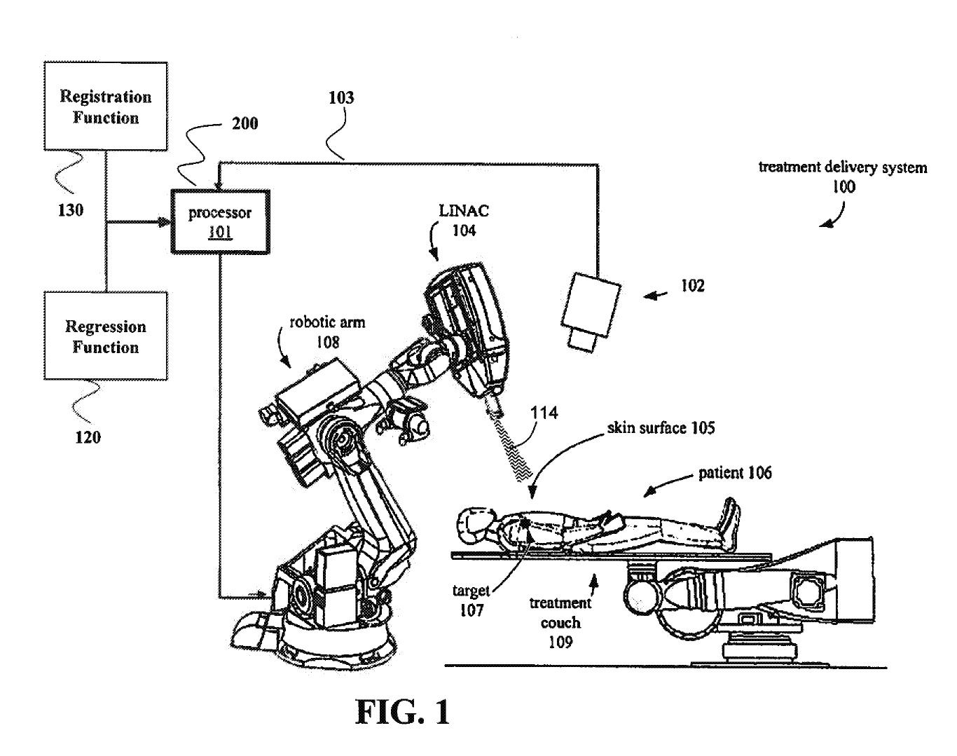

FIG. 1 shows a treatment delivery system 100 for performing radiotherapy of a target object, e.g., a tumor, in a body of a patient. It is understood that the radiotherapy can be performed with a variety of treatment modalities.

The treatment delivery system 100 determines a location of the tumor during various cycles of the respiration of the patient using a correspondence between a motion of the skin and a motion of the tumor. The correspondence is established during treatment planning sessions using a regression function and a registration function, as described in more details below. In one embodiment, the regression function and/or registration function are updated during the treatment preparation session.

The treatment delivery system 100 includes a motion tracking system 200 employing the principles of the invention using a processor 101, a motion sensor 102 for acquiring a motion signal 103 of a moving skin surface 105 of the patient 106. The treatment delivery system 100 can also include a linear accelerator (LINAC) 104, and a robotic arm 108. The motion tracking system 200 tracks the motion of the tumor 107 of the patient 106 during a treatment delivery session, while the patient lies on a treatment couch 109.

The motion tracking system 200 is operatively connected to the motion sensor 102 for acquiring the motion signal 103. In one embodiment, the processor 101 is configured to control operation of the motion sensor 102. Additionally or alternatively, the operation of the motion sensor 102 can be predetermined or controlled by another processor. The motion sensor 102 can be any device capable of acquiring data that can be used to produce the motion signal 103 of the motion of the skin surface 105. For example, in one embodiment the motion sensor 102 is a laser scanner or a photogrammetry system.

The motion tracking system 200 uses the regression function 120 and the registration function 130. The regression function 120 is used to determine a feature signal based on the motion signal 103. The feature signal represents a medical image of the body of the patient, e.g., two-dimensional (2D) imaging data such as an x-ray image. The regression function 120 is trained during the treatment planning session based on a set of observations of the motion signal 103 synchronized with a set of feature signals. In one embodiment the medical image is the x-ray image, and the feature signal is extracted from the x-ray images. The trained registration function is stored in a computer-readable medium.

Examples of the feature signal are appearance and statistics based descriptors, pixel intensities, intensity histograms, histogram of oriented gradients (HoGs), feature covariance descriptors, first and higher order region statistics, principal components or independent components of the x-ray image, frequency transforms, e.g., Fourier, discrete cosine, and wavelet transforms, and eigenfunctions.

In one embodiment, the feature signal is determined from the x-ray image. In one embodiment, the feature signal is determined from a pair of orthogonal x-ray images. The regression function 120 can be stored in any computer-readable medium, e.g., a memory of the system 200.

Similarly, the registration function 130 is determined in advance during the planning session and stored in the computer-readable medium. The registration function 130 is trained by mapping each feature signal in the set of feature signals to three-dimensional (3D) imaging data. The 3D imaging data can be 3D computer tomography (3D CT) data, or any other diagnostic imaging data. The 3D imaging data are acquired under breath-hold procedure, and has a location of the tumor identified for the treatment.

The registration function 130 is used to register each feature signal with the 3D imaging data of the patient.

The motion tracking system 200 can also be connected to an accelerator, e.g., a linear accelerator (LINAC) 104, which is capable of producing a particle beam suitable for radiotherapy. In one embodiment, the motion tracking system 200 is connected to the LINAC 104 such that the processor 101 controls the beam and other aspects of operation of the LINAC 104. The processor 101 can also be configured to receive information from the LINAC 104, such as status information. LINAC 104 can be mounted on a robotic arm 108, which is be controlled by processor 101. The robotic arm 108 can direct the processor 101 to direct the beam 114 of the LINAC 104 at different locations and from different angles.

In one embodiment, after determining the location of the tumor, the motion tracking system 200 issues commands to move the robotic arm 108, such that the beam of LINAC 104 intersects the target object 107. In some embodiments, the target object 107 is a pathological anatomy such as the tumor. Alternatively, target 107 can be any subject for which location tracking is desired. By repeating the process of acquiring the motion signal 103, determining the feature signal and registering the feature signal, and moving the robotic arm 108 such that the beam of LINAC 104 intersects with tumor, the treatment delivery system 100 track the location of tumor continuously and maintain the beam of LINAC 104 directed at the tumor for the duration of the treatment delivery session, even while the tumor is moving.

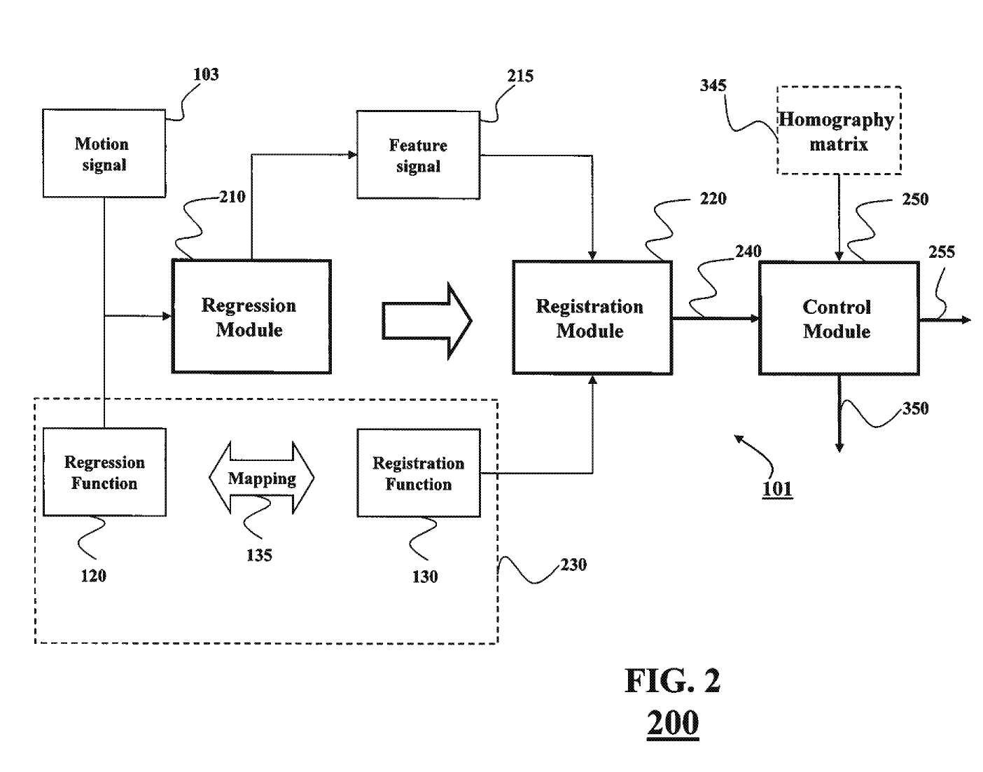

FIG. 2 shows a system 200 for determining a location 240 of the tumor based on the motion of the skin surface 105 according to embodiments of the invention. The system 200 is implemented using the processor 101, and can include a memory and an input/output interfaces as known in the art. The system 200 facilitates operation of the treatment delivery system 100 during the treatment delivery session.

A regression module 210 determines a feature signal 215 of two-dimensional (2D) imaging data of the body of the patient based on the motion signal 103 received from the motion sensor 102 and the regression function 120. In one embodiment, the regression function 120 is trained during the treatment planning session, as described in more details below. The regression function 120 is stored in a computer-readable medium 230 operatively connected to the regression module 210.

A registration module 220 determines the location 240 of the tumor using the registration function 130 by registering the feature signal 215 with three-dimensional (3D) imaging data of the patient, wherein a breath-hold location of the tumor in the 3D imaging data is identified. Specifically, the registration function 130 registers each feature signal 215 to a digitally reconstructed radiograph (DRR) image of the 3D imaging data. In one embodiment, the registration function 130 is trained during the treatment planning session and stored in the computer-readable medium 230 operatively connected to the regression module 210.

A control module 250 generates a command 255 for controlling an operation of components of the treatment delivery system 100. For example, the control module 250 can generate a command 255 for moving the robotic arm 108 and/or the LINAC 104 of the treatment delivery system 100.

In one embodiment, the control module 250 generates the command 255 based on the location 240 of the tumor in coordinates relative to the 3D imaging data. However, during a treatment preparation session, the patient is aligned on a treatment couch such that position of the patient is aligned with the previously taken 3D imaging data. Typically, this alignment is represented by a homography matrix 345. Accordingly, in one embodiment, the control module 250 generates the command 255 based on a global location 350 of the tumor determined based on the location 240 and the homography matrix 345.

In one embodiment, the regression and the registration functions are combined into a mapping function 135. The mapping function 135 combines the registration and the regression function into a single function, which provides the correspondence between the motion signal and the location of the tumor.

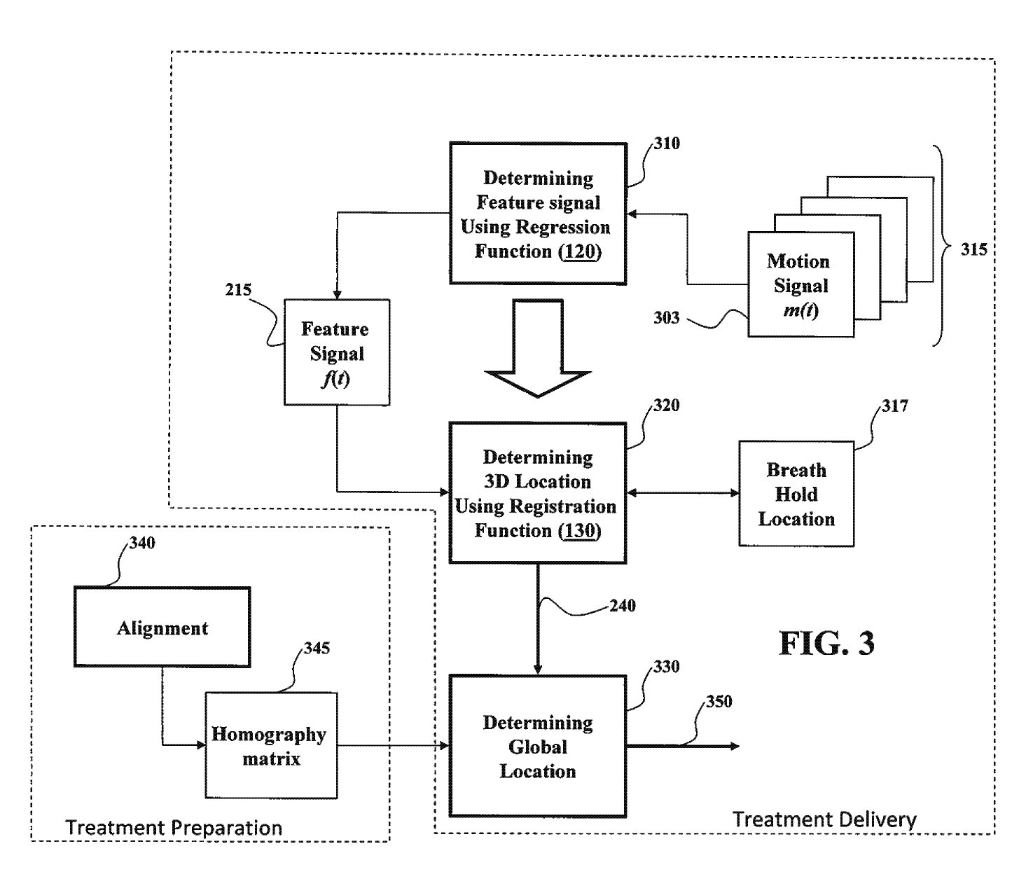

FIG. 3 shows a block diagram for determining the global location 350 of the tumor based on the motion signal 103. Sample observations 315 of the motion signal 103 are acquired during time t of the treatment delivery session. The observations 315 are acquired with a predetermined frequency. The regression function 120 provides the correspondence between the observations 315 of the motion signal and corresponding feature signals 215. Because the feature signals 215 include the tumor at different locations, the regression function 120 allows tracking motion of the tumor in the feature signals 215 with the motion of the skin of the patient.

Similarly, the registration function 130 registers locations of the tumor in the feature signals 215 to the breath-hold location 317 of the tumor in the DRR image and/or the 3D imaging data. Hence, the registration function 130 determines a location of the tumor in a particular feature signal 215 with respect to the breath-hold location 317 in coordinates of the 3D imaging data. By concatenation effects of the regression and the registration functions, the motion of the tumor in coordinates of the 3D imaging data is tracked based on the motion of the skin of the patient.

Specifically, the particular feature signal 215 is determined 310 for the observation 303 of the motion signal using the regression function 120. The feature signal 215 is mapped to the breath-hold location 317 of the tumor by the registration function determining 320 the location 240 of the tumor corresponding to the observation 303.

During the treatment preparation session, the patient is typically aligned by an alignment module 340 on the treatment couch 109, such that global location of the breath-hold location is determined. For example, a homography matrix 345 is determent as a result of the alignment, which maps breath-hold location of the tumor in coordinates of the 3D imaging data with global coordinates utilized by the treatment delivery system. Thus, one embodiment of the invention determines 330 the global location 350 of the tumor based on the location 240 and the homography matrix 345.

Motion Signal

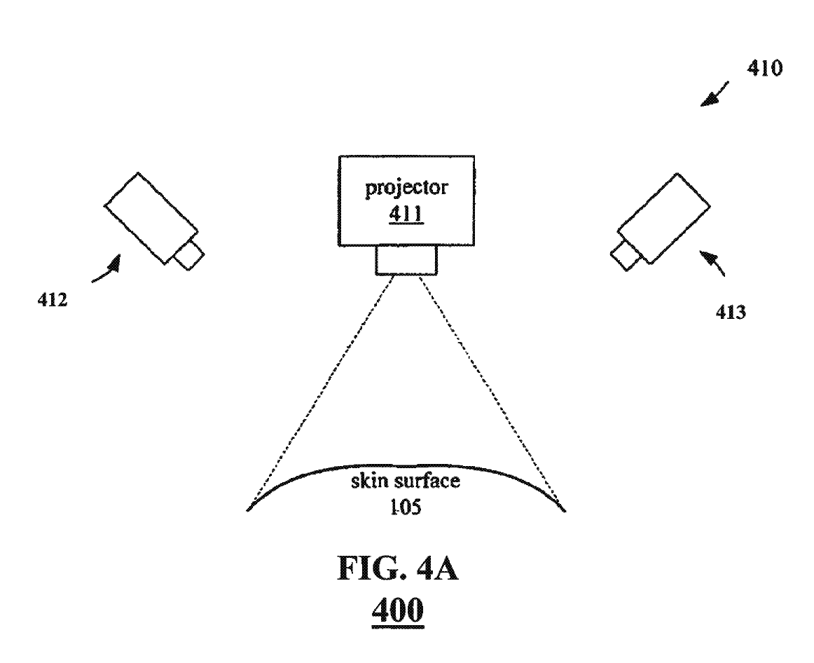

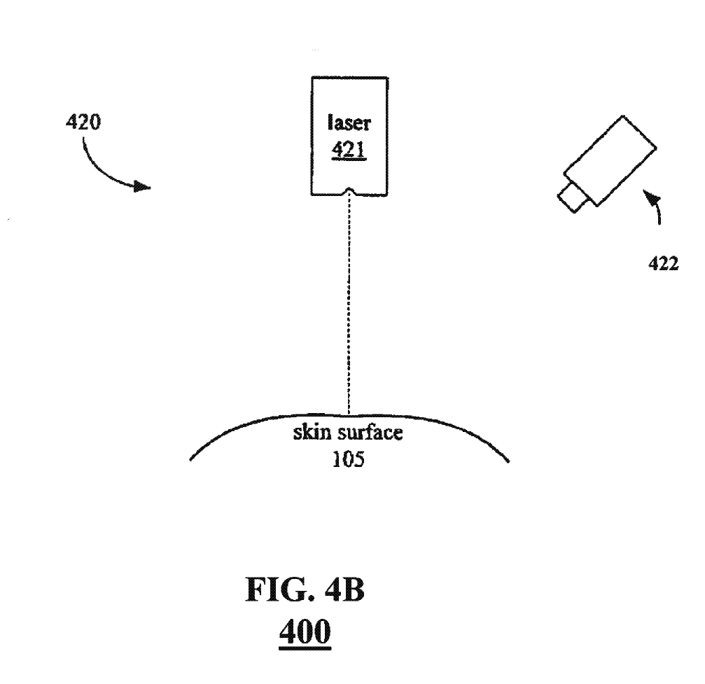

FIGS. 4A-B show examples of determining 400 the motion signal of the motion of the skin surface 105 of the patient. In those examples the motion sensor 102 is a laser scanning system 420 or a digital photogrammetry system 410.

For example, FIG. 4A shows components of a digital photogrammetry system that is used as the motion sensor 102, according to one embodiment of the invention. Digital photogrammetry system 410 includes projector 411 and cameras 412 and 413. The digital photogrammetry system projects light onto the skin surface 105 using the projector 411. In one embodiment, the projector 411 projects a pattern, such as a pattern of evenly spaced dots onto the skin surface 105.

Alternatively, different types of patterns, such as lines or a grid, can also be projected on the skin. While the pattern is projected, cameras 412 and 413, arranged at different angles with respect to the skin surface 105, acquire images of the skin surface 105 by acquiring light reflected from the skin surface 105. The images of the skin surface 105 are used to triangulate positions of points on the skin surface 105. The images are taken over the time with a specified periodicity, such that the motion signal for each point is determined.

In an alternative embodiment, the motion sensor 102 is the laser scanning system 420 shown in FIG. 4B. The laser scanning system 420 includes a laser 421 and a camera 422. The laser projects a laser beam 430 in a predetermined direction to the skin surface 105. The camera 422 acquires the location of the resulting point of the laser beam 430 reflected by the skin surface 105. Subsequently, the laser 421 projects the laser beam 430 onto the same and/or a different location on the skin surface 105, after which the camera 422 can again acquire the location of the point of the laser beam 430. The location of each of these points in three-dimensional space are be triangulated using the known direction of the projected laser beam and the location of the point as acquired by the camera 422. The process is repeated over time to determine the motion signal for every desired point on the skin surface 105.

Other embodiments use different methods to acquire the motion signal of the skin. For example, the motion signal can be acquired using a method similar to time-of-flight laser range finding. Another embodiment uses a stereoscopic camera system to illuminate the skin with special patterns and the motion signal is constructed from these observations. Alternatively, 3D rangers, optical stereo, ultra-sound and multi-cameras, and other structured light devices can be used to obtain the motion signal.

During the determination of the motion signal, the skin surface 105 moves during the respiratory cycle of the patient 106. Thus, the motion signal tracks the respiration cycle of the patient.

Regression Function

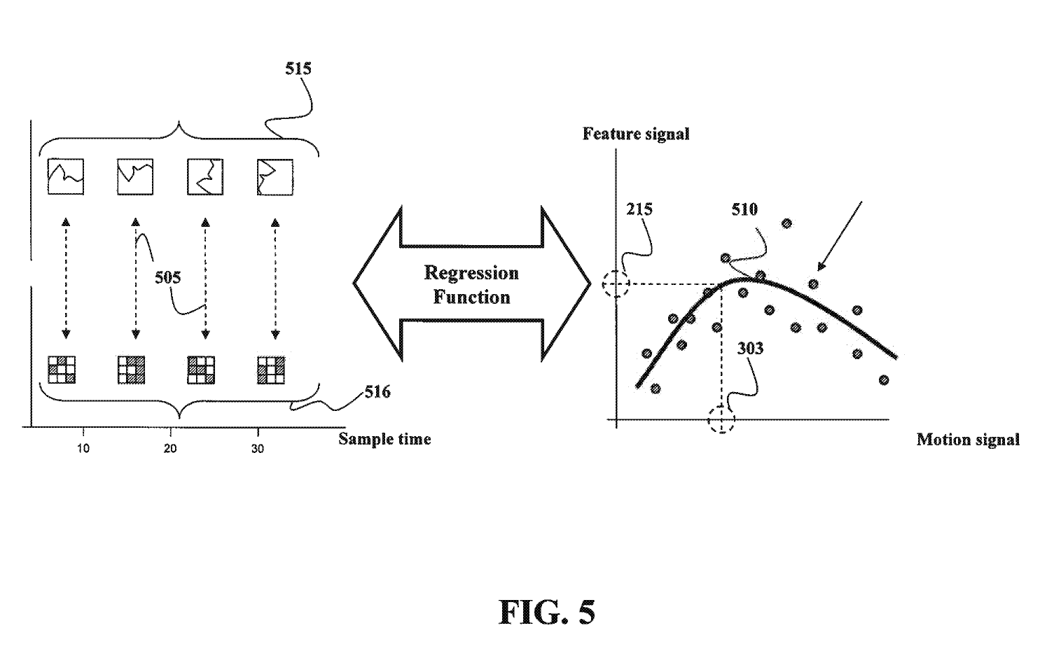

As an example, FIG. 5 shows a schematic of training the regression function 120. The regression function is trained during the treatment planning session. As shown in FIG. 5, the regression function establishes a correspondence between the motion signal and the set of the feature signals. Specifically, the regression function establishes the correspondence 510 between the set of observations 515 of the motion signals and the set 516 of the feature signals.

The sets 515 and 516 do not have to be continuous, but elements of the sets are synchronized 505 with each other. Knowing the regression function, during the treatment delivery session, the particular feature signal 215 can be determined from the particular observation of the motion signal 303. The feature and the motion signals can be of any dimensions. The regression function 120 can be any complex function that has the best fit to the series of observation pairs between the feature signal and motion signal.

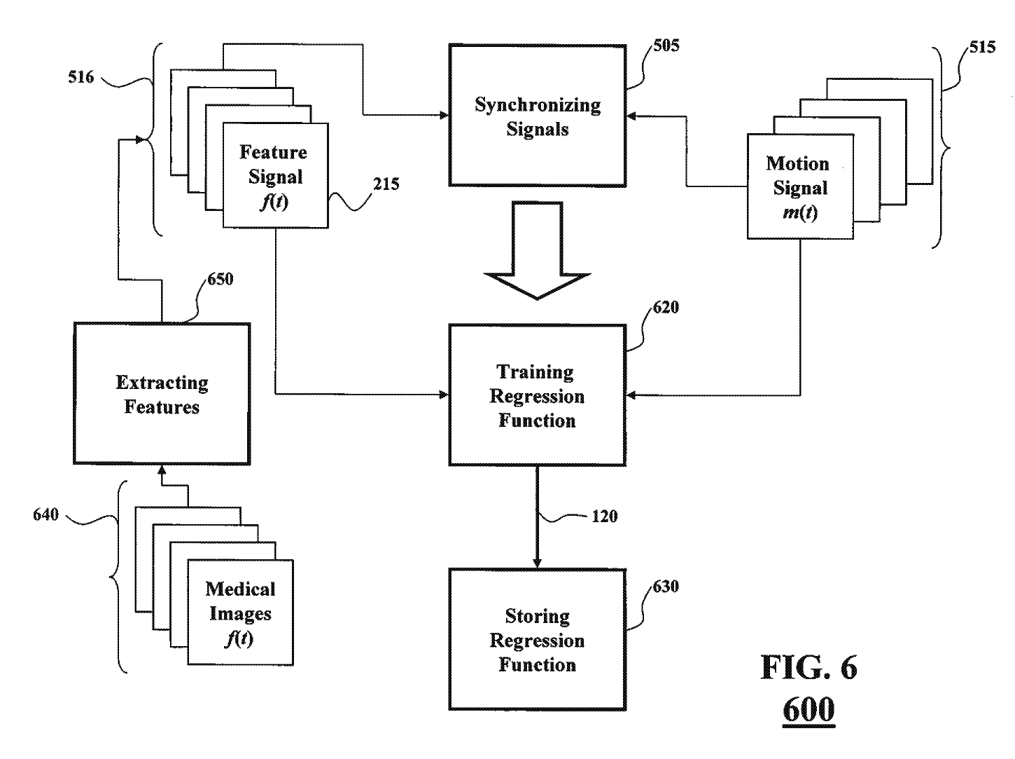

FIG. 6 shows a block diagram of a method 600 for determining the regression function 120. During the treatment planning session, a set of medical images 640 and corresponding set 515 of the observations of the motion signals are acquired simultaneously such that the sets 640 and 515 are synchronized 505 in time. In one embodiment, each medical image includes a pair of medical images acquired for different angles to capture 3D position of the tumor. In one embodiment, the medical images of the pair are orthogonal to each other. The set 515 of observations of the motion signal is generated by observing, e.g., the parts of the chest and abdominal area. At each specified time instant, the observation of the motion signal is determined representing, e.g., relative 3D location of the point on the skin. Additionally or alternatively the motion signal can be represented by 2D and 3D motion parameters.

In one embodiment, for each medical image in the set 640, the feature signal is determined 650 to form the set of feature signals 516. Because the sets of medical images 640 and observations of the motion signal 515 are synchronized in time, the set of feature signals 516 is also synchronized with the set of observations 515. Typically, the feature signal has a smaller dimension than the corresponding medical image. However, in one embodiment the medical images are used as the feature signals. Various embodiments of the invention use dimensionality reduction methods, such as principal component analysis (PCA), Fisher discriminant analysis (FDA), clustering, a histogram of oriented gradients or intensity change methods, to represent the medical image as the feature signal.

In one embodiment, the medical image is the x-ray image. In alternative embodiments, the medical image is any medical images that can identify the tumor. Examples of such medical images include ultrasound images, magnetic resonance imaging (MRI), positron emission tomography (PET), single photon emission computed tomography (SPECT), and photo-acoustic tomography (PAT) images, and digital thermograph imaging,

The regression function is trained 620 by using corresponding pairs of features and motion signals. Examples of the training methods includes, but are not limited to, a polynomial regression, a nonlinear regression, a nonparametric regression methods and/or methods that use splines. The trained regression function 120 is stored 630 on the computer-readable medium.

Forms of Regression Functions

Regression analysis is the problem of estimating an average level of a quantitative response variable from various predictor variables. A regression function fits the model

yᵢ=f(xᵢ)+εᵢ

at a representative range of values of x, which are the n observations xᵢ considering errors εᵢ.

Linear regression is a common quantitative tool. However, there are very few situations wherein usage of the linear regression can be justified. Therefore, some embodiments of the invention use nonlinear regression.

Nonparametric regression analysis is regression without an assumption of linearity. The scope of nonparametric regression is very broad, ranging from “smoothing” the relationship between two variables in a scatterplot to multiple-regression analysis and generalized regression models, e.g., logistic nonparametric regression for a binary response variable.

Polynomial Regression Function:

Polynomial regression performs a p^{th}-order weighted-least-squares of y on x,

yᵢ=b₀+b₁(xᵢ−x₀)+b₂(xᵢ−x₀)²+ . . . +bₚ(xᵢ−x₀)^{p}+εᵢ,

and weighting the observations in relation to their proximity to the focal value x₀ within a window enclosing the observations for the local regression. A vector b=(b₁, . . . , bₚ) is a vector of parameters to be estimated, and xᵢ=(x₁, . . . , xₖ) is a vector of predictors for the i^{th} of n observations. Errors εᵢ are normally and independently distributed with a mean 0 and a variance σ². One embodiment adjusts the window size h so that each local regression includes a fixed proportion of the data. The proportion is a span of the local-regression smoother.

Nonlinear Regression and Nonparametric Regression Functions:

The nonlinear regression model fits the model

yᵢ=f(b,xᵢ)+εᵢ.

The function f(•) relates the average value of the response y to the predictors.

The general nonparametric regression model is written in a similar manner, but the function ƒ is unspecified:

yᵢ=f(xᵢ)+εᵢ=f(xᵢ₁,xᵢ₂, . . . , xᵢₖ)+εᵢ

The object of nonparametric regression is to estimate the regression function f(•) directly, rather than to estimate the parameters. Most methods of nonparametric regression implicitly assume that f(•) is a smooth, continuous function. An important special case of the general model is nonparametric simple regression, where there is only one predictor:

yᵢ=f(xᵢ)+εᵢ.

Nonparametric simple regression is defined as scatter plot smoothing. One restrictive nonparametric regression model is the additive regression model

yᵢ=f₀+f₁(xᵢ₁)+f₂(xᵢ₂)+ . . . +fₖ(xᵢₖ)+εᵢ,

where the partial-regression functions fₖ(•) are assumed to be smooth, and are to be estimated from the data. This model is more restrictive than the general nonparametric regression model, but less restrictive than the linear regression model, which assumes that all of the partial-regression functions are linear. Variations on the additive regression model include semi-parametric models, in which some of the predictors enter linearly, and models in which some predictors enter into interactions, which appear as higher-dimensional terms in the model. Some other nonparametric regression models include projection-pursuit regression, and classification and regression trees.

Nonparametric regression techniques can smooth observed data corrupted by some level of noise. A subset of these techniques is based on defining an appropriate dictionary of basis functions from which the final regression model is constructed. The model is usually defined to be a linear combination of functions selected from the dictionary.

Some embodiments of the invention assumed that the motion signal is a linear combination of the selected basis functions. The main problem associated with this approach is the appropriate definition of the dictionary and the selection of a subset of basis functions used for the final model. Using a fixed dictionary of several basis functions, for example, all polynomials up to a pre-defined order or several trigonometric functions, can provide an easier selection among basis functions, but in general does not guarantee the possibility to closely approximate the motion signal. Defining the solution in a functional space can guarantee exact functional approximation of the motion signal.

Spline Regression Function:

Splines are a solution to the regression problem of determining the function f(x) with two continuous derivatives that minimizes the penalized sum of squares,

Σ[yᵢ−f(xᵢ)]²+h∫[fₓₓ(x)]²dx,

where h is a smoothing parameter, analogous to the neighborhood-width of the local-polynomial estimator. The first term is the residual sum of squares. The second term is a roughness penalty, which is large when the integrated second derivative of the regression function fₓₓ(x) is large, i.e., when the function f(x) rapidly changes slope. At one extreme, when the smoothing constant h is zero and all the x-values are distinct, the function f(x) interpolates the data. At the other extreme, if h is very large, then the function f is selected so that the regression function fₓₓ(x) is zero everywhere, which implies globally linear least-squares fit to the data. The function f(x) that minimizes the penalized sum of squares is a natural cubic spline with knots at the distinct observed values of x.

Registration Function

Each of the feature signal acquired during the treatment planning sessions is registered to the coordinate system of the 3D imaging data. From multiple registrations, the registration function that registers the set of feature signals to the 3D imaging data is determined. The registration function is also determined during the treatment planning session.

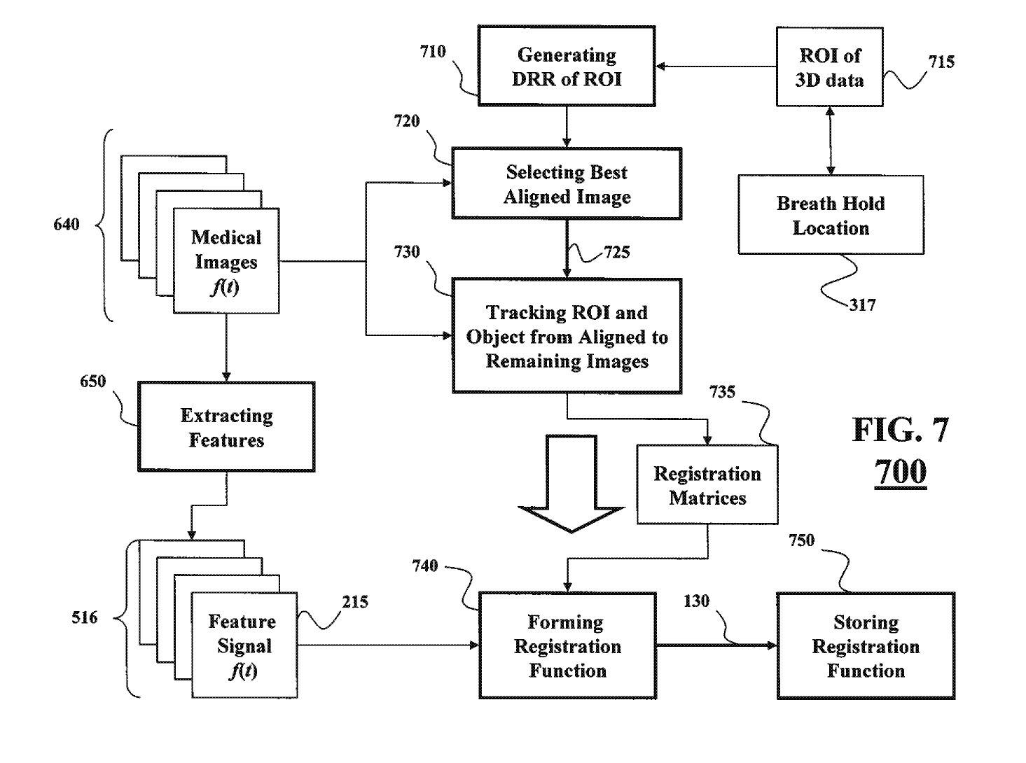

FIG. 7 shows a block diagram of a method for determining the registration function 130 according one embodiment. A digitally reconstructed radiograph (DRR) is obtained from a region of interest (ROI) 715 of the 3D imaging data. The ROI of the DRR includes the tumor. The 3D imaging data are acquired during the breath-hold procedure. The location 317 of the tumor at the breath-hold is identified, e.g., manually by medical professional, or automatically using conventional recognition methods. However, during different stages of the respiration of the patient, the location of the tumor differs from the location at the breadth-hold.

The set 640 of medical images is taken concurrently with the motion signal during the treatment planning session, as described above. For example, the set 640 of medical images can be acquired with a periodicity of 30 frames per second, and can include several hundreds of images. Each of the medical images is compared with the DRR image to select 720 the medical image 725 that is best aligned with the DRR image.

Next, using any tracking method, pixels of each medical image are tracked from the best aligned image to determine a set of registration matrices 735. Each registration matrix in the set 735 maps the corresponding medical image to the best aligned image, and respectfully, to the DRR image. Thus, the registration matrix enables two-way transformation mapping between the medical image and the DRR image. In one embodiment, the tracking is restricted only to the ROI.

As described above, in some embodiment, the feature signals are extracted from the medical images to reduce the amount of data. Thus, the set of feature signals 516 and/or corresponding registration matrices form 740 the registration function 130. The registration function is stored 750 in the computer-readable memory for subsequent use during the treatment sessions.

Alignment

During each treatment preparation session, one or several pairs of orthogonal x-ray images are taken to align the patient on the treatment couch such that the target object is under a target radiation area. Typically, the alignment is performed by an alignment module registering the pairs of X-ray images with the DRR image.

In one embodiment, the alignment procedure produces the homography matrix that transfers the coordinates of the 3D imaging data to global coordinates based on the global coordinates of the pair of the X-ray images. In one variation of this embodiment, the registration function is updated based on the homography matrix to register each feature signal to the global coordinates. Because the location of the tumor is already mapped to the 3D imaging data, by applying the registration function to the feature signals, the global location of the target object is determined.

Update Module

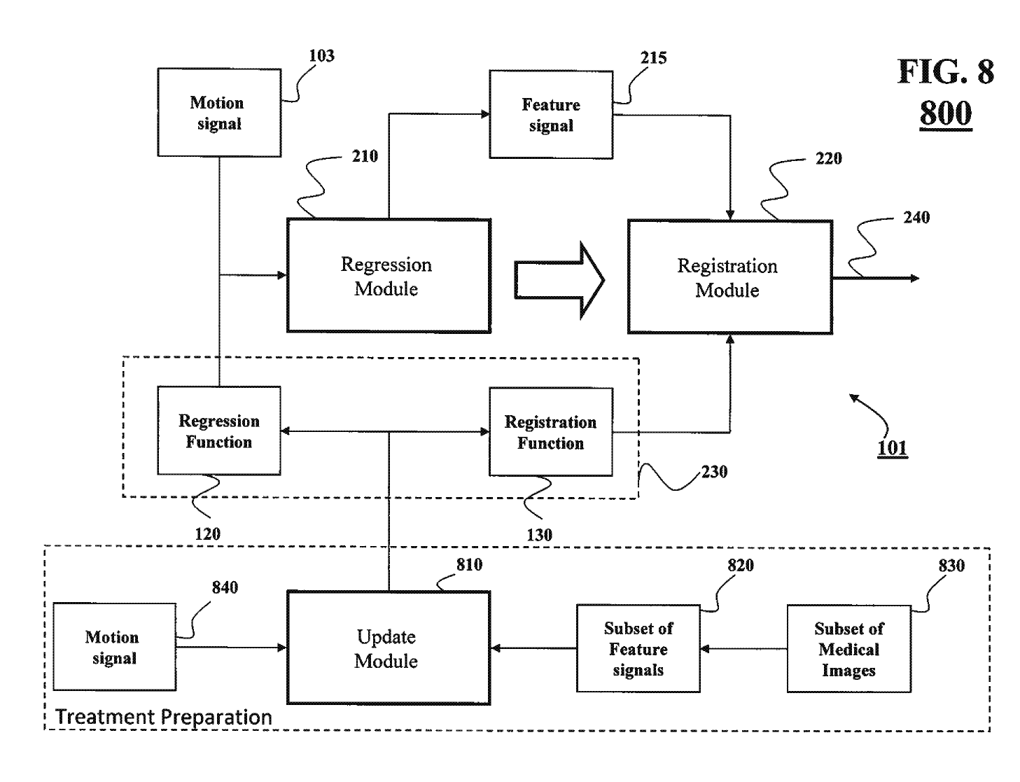

FIG. 8 shows another embodiment of the invention, which is based on a realization that correspondences between the motion of the skin and the location of the tumor can be updated during the treatment using medical images having a quality lower than required to determine the correspondence. Thus, the treatment time and the risk of potential harm are reduced without compromising the quality of the correspondence.

During the treatment preparation session, a few synchronized low-dosage medical images, e.g., x-ray images, and motion signals are acquired. Using the feature signals determined for the low-dosage medical images and the respective motion signals, the regression and/or the registration function are updated.

Accordingly, one embodiment includes an update module 810 for updating the regression function 120 and/or the registration function 130. The update module 810 updates the regression and the registration functions based on the motion signal 840 and a subset 820 of feature signals acquired during the treatment preparation session. In one embodiment, each medical image in the subset is a pair of medical images, e.g., orthogonal medical images. The subset 820 of feature signals represents medical images 830. In one embodiment, each feature signal in the subset 820 of feature signals that is extracted from a low-dosage medical image 830, i.e., a resolution of the medical images 830 is less than a resolution of the medical images 640. Additionally or alternatively, a size of the subset 820 is less than a size of the set 516.

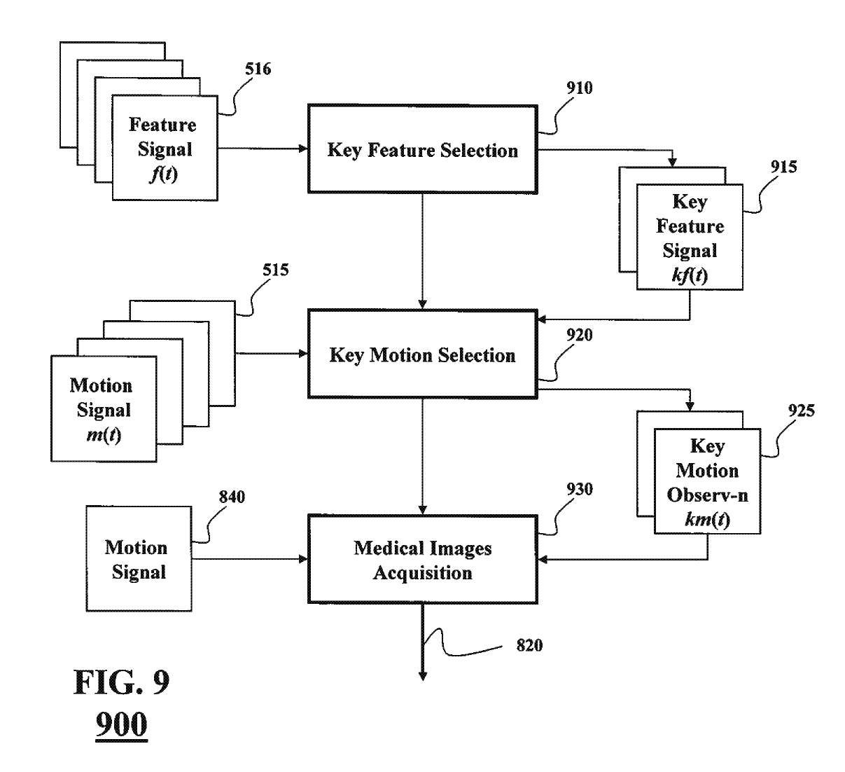

FIG. 9 shows an example of determining the subset of feature signals according to one embodiment of the invention. From the set 516 of the feature signals, a set of key feature signals 915 is selected 910 such that the position of the tumor in the set 915 can be interpolated. For example, each feature signal in the set 915 represents different position of the tumor, and thus defines a time instant of the possible motions of the tumor. A set of key motion observations 925 of the motion signal corresponding to the set of the key feature signals 915 is selected from the set 515 of observations of the motion signal. Accordingly, during the treatment preparation session, the set of key motion observations 925 is determined based on the sets of feature signals and motion observations acquired during the treatment planning session.

Next, when value of the motion signal matches with one of the key observations from the set 925, a medical images acquisition device is triggered to acquire 930 the subset of medical images 830, from which the subset of feature signals 820 is extracted.

Accordingly, the feature signals in the subset 820 corresponds to the key feature signals in the subset 915 and reflect recent changes in the position of the tumor since the treatment planning session due to, e.g., changes in weight of the patient and an internal motion of fluid. Because the subset of medical images is acquired only according to the key motion observations, the amount of additional radiation during each treatment planning session is drastically reduced.

For example, instead of acquiring 30 seconds of continuous 30 frames-per-second (fps) video of medical images, which accumulates 900 x-ray images or 1800 orthogonal x-ray images, some embodiments acquire only 9 low-dosage images or 18 orthogonal x-ray images, each low-dosage images corresponds to the key observation of the motion signal. Such low number of medical images corresponding to the key observation of the motion signal is sufficient to update the regression function with almost the same quality as with conventional 1800 images. Hence, the update module significantly reduces the risk of potential harm to the patient caused by exposure to the radiation.

The key motion observations correspond to the specific instances of the motions of the tumor. One key motion observation corresponds to specific position of the tumor, two key motions observations correspond to significantly different positions of the tumor, and the set of key motions motion observations correspond to all significantly different positions of the tumor.

As described above, one embodiment of the invention determines the key motion observation as observations of the motion corresponding to the key feature signals representing different positions of the tumor. One variation of this embodiment determines the set of key feature signals by clustering the feature signals, such that one cluster of feature signals corresponds to a key feature signal.

One way to obtain the key motion observations is to cluster the motion signals. In addition to the motion signal, the associated feature signals can be clustered, and the key feature signals are obtained by clustering the feature signals. Examples of clustering techniques include k-means, k-medoids, Delaunay triangularization, spectral clustering, e.g., eigenvector projection, principal component analysis (PCA)m mode seeking by kernel updates, density estimation, model fitting by expectation maximization, an other techniques. Additionally or alternatively, one embodiment of the invention clusters the set of motion observation determined during the planning treatment session.

Regression Function Update

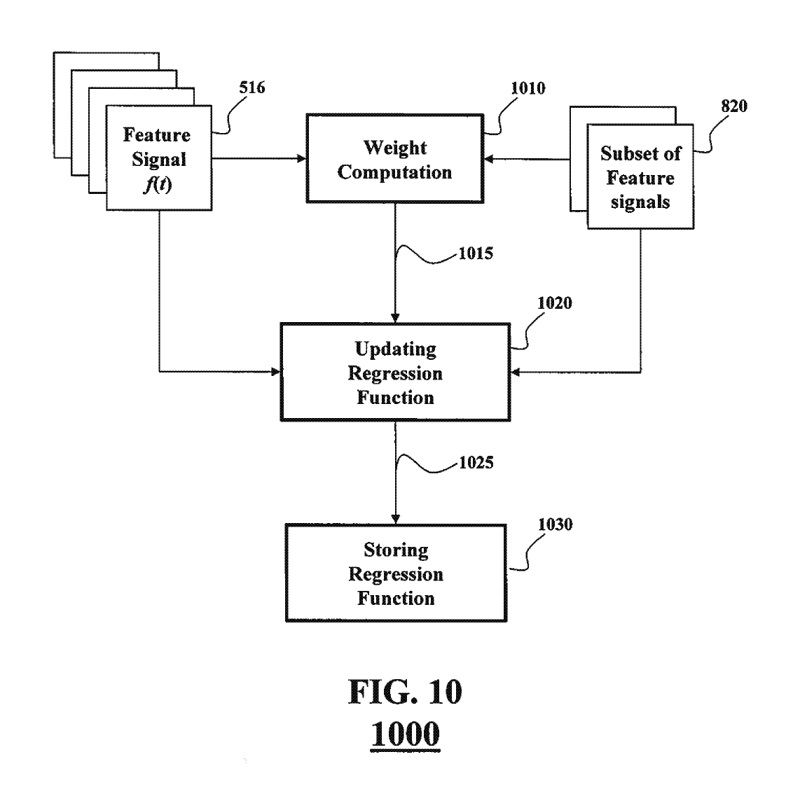

FIG. 10 shows an example of updating the regression function based on the subset of feature signals. The subset of feature signals 820 is compared with the set of feature signals 516. Individual weights are assigned 1010 to each feature signal to adjust the contribution of the corresponding feature signal. For instance, more recently determined feature signals are given higher weights to adapt for recent changes of the position of the tumor. In one embodiment, the sum of the weights is equal to 1. The feature signals and the corresponding motion signals are used to update 1020 the original regression function. During the update, each motion signal is weighted by an assigned weight 1015. The updated regression function 1025 is stored 1030 in the memory.

Various embodiments of the invention update the regression function differently. For example, the polynomial regression function can be expressed by a data matrix X, a response vector Y, and a parameter vector f. The i^{th} row of data matrices X and Y includes the x and y value for the i^{th} data sample. The model can be expressed as a system of linear equations:

Y=fX+ε

and the vector of estimated polynomial coefficients is

f=(X^{t}X)⁻¹X^{T}Y.

The polynomial coefficient estimates are calculated by setting the ε=0 and solving the system of linear equations provided the number of parameters is smaller than the number of motion-feature signal pairs.

Alternatively, the updating of the regression function is done by selecting a window size, which can be adaptive to data as a window including a certain number of nearest neighbors of center point x₀; assigning weights to each observations in the neighborhood of x₀, locally fitting a weighted regression line to the data in the neighborhood of x₀, which means a local polynomial regression of order p=1; and combining the local regressions for a range of x-values.

Registration Function Update

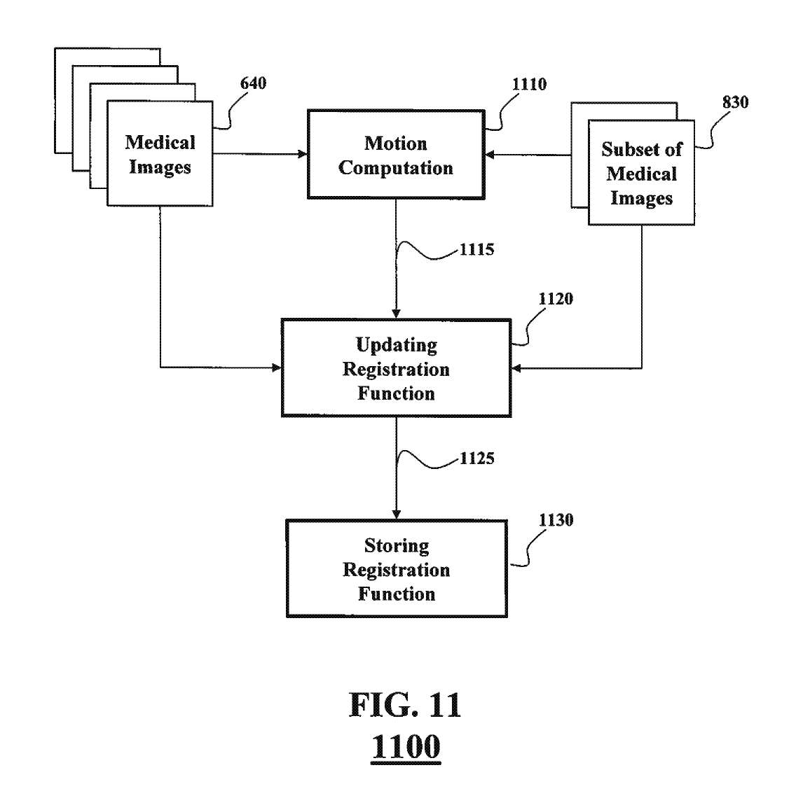

FIG. 11 shows a method for updating the registration function based on motions 1115 of the tumor in the subset of medical images compared 1110 with the set of medical images. The motion can be defined as the pixel-wise dense optical flow, block-wise motion vector, or image subtraction. One embodiment determines the motions between the medical images in the subset 830 of medical images and corresponding most similar medical images in the set 640 of medical images. The most similar medical images have the smallest feature signal distance or the smallest motion signal distance between each other.

The motion 1115 is used to determine registration matrices, and the registration function is updated 1120 using all previous and currently determined registration matrices and the corresponding feature signals.

The embodiments adapt the internal regression and registration functions to the inevitable changes that happen between the consecutive treatment sessions to achieve the most accurate tumor positioning and tracking.

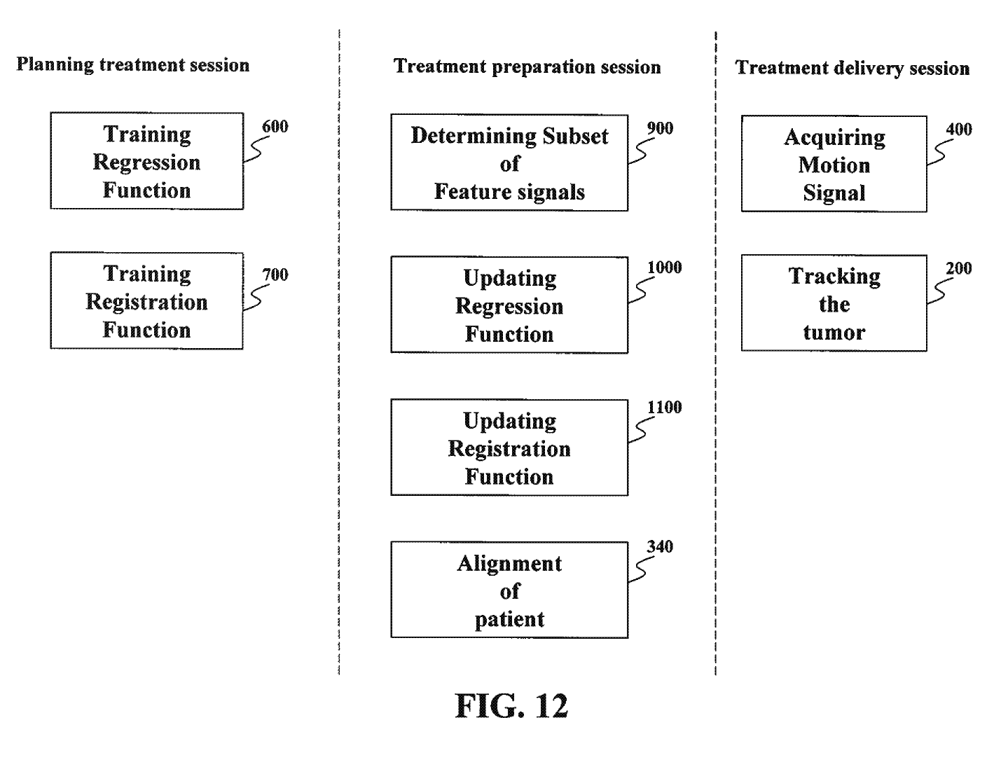

FIG. 12 shows examples of different procedures from which the embodiments of the invention take an advantage. For example, the training 600 of regression function and the training 700 of the registration function can be performed during the planning treatment session and reuse multiple time for tracking 200 the tumor during the treatment delivery session.

During the treatment preparation session, the patient is aligned 340, and the regression function and the registration function can be updated 1000 and 1100 using the subset of feature signals. Determination 900 of the subset of feature signal is less harmful for the patient than, e.g., the conventional 4D data model determination performed for each treatment.

During the treatment delivery session, the motion of the tumor is determined by tracking 400 the motion of the skin of the patient. Accordingly, the embodiments of the invention provide a method and a system for determining a location of a tumor in a body of a patient based on a motion of the skin of the patient during any phase of the respiration of the patient, while minimize exposure of the patient to the unhealthy body imaging and without using invasive fiducial markers. Thus, the treatment time and the risk of potential harm to the patient are reduced without compromising the quality of the treatment.

In these embodiments, the real-time tumor positioning enables continuous treatment of the tumor, thus effectively shortening duration of the treatment delivery sessions. As a result, the embodiments make the most economical and practical use of the limited availability of the particle beam treatment centers.

Although the invention has been described by way of examples of preferred embodiments, it is to be understood that various other adaptations and modifications may be made within the spirit and scope of the invention. Therefore, it is the object of the appended claims to cover all such variations and modifications as come within the true spirit and scope of the invention.

I claim:

1. A system for facilitating an operation of a treatment delivery system based on a location of a target object in a body of a patient, wherein the location is subject to a motion caused by respiration of the patient, comprising:
a regression module for selecting, using a motion signal and a regression function, a feature signal from a set of feature signals, each feature signal in the set of feature signals represents a medical image of the body of the patient, wherein the motion signal represents a motion of a surface of a skin of the patient caused by the respiration, and wherein the regression function is trained based on a set of observations of the motion signal synchronized with the set of feature signals;
a registration module for determining the location of the target object using the feature signal and a registration function, wherein the registration function registers each feature signal to a three-dimensional (3D) imaging data having a breath-hold location of the target object identified; and
a control module for generating a command to facilitate the operation of the treatment delivery system based on the location.

2. The system of claim 1, further comprising:
an update module for updating the regression function and the registration function based on a subset of feature signals representing a subset of medical images.

3. The system of claim 2, wherein a size of a subset of the feature signals is less than a size of the set of the feature signals,

4. The system of claim 3, wherein each feature signal in the subset of feature signals is extracted from a low-dosage medical image.

5. The system of claim 2, wherein each feature signal in the subset of feature signals is acquired when an observation of the motion signal matches an observation from a set of motion observations, wherein each motion observation from the set of motion observations corresponds to a feature signal from a set of key feature signals representing different locations of the target object.

6. The system of claim 1, further comprising:
an alignment module for updating the registration function with global coordinates of the breath-hold location of the target object.

7. The system of claim 2, wherein the regression function and the registration function are determined during a planning session, and wherein the regression function and the registration function are updated during the treatment session.

8. The system of claim 1, wherein the 3D imaging data acquired during a planning session using a breath hold procedure.

9. The system of claim 1, further comprising:
a processor for controlling the treatment delivery system, such that a beam of radiation is directed at the location of the target object.

10. The system of claim 1, further comprising:
a motion sensor for determining the motion signal.

11. A method for controlling an operation of a treatment delivery system such that a beam of radiation is directed at a target object in a body of a patient for a duration of treatment session, wherein a location of the target object is subject to a motion, comprising the steps of:
selecting, using a motion signal and a regression function, a feature signal from a set of feature signals, each feature signal in the set of feature signals represents a medical image of the body of the patient, wherein the motion signal represents a motion of a surface of a skin of the patient caused by the respiration, and wherein the regression function is trained based on a set of observations of the motion signal synchronized with the set of feature signals;
determining the location of the target object using the feature signal and a registration function, wherein the registration function registers each feature signal to a digitally reconstructed radiograph (DRR) image having a breath-hold location of the target object identified; and
controlling the operation of the treatment delivery system based on the location, such that a beam of radiation is directed at the location of the target object.

12. The method of claim 11, further comprising:
updating the regression function during the treatment session.

13. The method of claim 11, further comprising:
updating the registration function during the treatment session.

14. The method of claim 11, further comprising:
updating the regression and the registration functions during the treatment session based on a subset of feature signals representing a subset of medical images representing different locations of the target object.

15. The system of claim 11, wherein the target object is a tumor, the medical image is an x-ray image, and the DRR image is determined from three-dimensional (3D) imaging data acquired using a breath hold procedure.

16. The method of claim 11, further comprising:
acquiring the set of medical images concurrently with the set of observations of the motion signal;
extracting the set of feature signals from corresponding medical images in the set of medical images; and
training the regression function based on corresponding pairs of the set of feature signals and the set of observations of the motion signal.

17. The method of claim 16, further comprising:
determining an aligned medical image that is best aligned with the DRR image;
tracking pixels in each medical images from pixels of the aligned image to determine a set of registration matrices; and
determining the registration function based on the set of registration matrices.

18. A system for facilitating an operation of a treatment delivery system based on a location of a tumor in a body of a patient, wherein the location is subject to a motion caused by respiration of the patient, comprising:
a regression module for selecting, using a motion signal and a regression function, a feature signal from a set of feature signals, each feature signal in the set of feature signals represents a medical image of the body of the patient, wherein the motion signal represents a motion of a skin of the patient caused by the respiration, and wherein the regression function is trained based on a set of observations of the motion signal synchronized with the set of feature signals;
a registration module for determining the location of the tumor using the feature signal and a registration function, wherein the registration function registers each feature signal to a breath-hold location of the tumor; and
an update module for updating the regression function and the registration function based on a subset of feature signals representing different locations of the tumor.

19. The system of claim 18, further comprising:
an alignment module for updating the registration function with global coordinates of the breath-hold location of the tumor;
a motion sensor for determining the motion signal; and
a processor for controlling the treatment delivery system, such that a beam of radiation is directed at the location of the target object.

20. The system of claim 1, further comprising:
means for determining the regression function and the registration function.
